(19) United States
(12) Patent Application Publication
(10) Pub. No.: US 2024/0316019 A1
(43) Pub. Date: Sep. 26, 2024

(54) METHODS OF PREVENTING SYMPTOMATIC ISCHEMIC STROKE

(71) Applicant: Bayer Aktiengesellschaft, Leverkusen (DE)

(72) Inventors: Hardi MUNDL, Esslingen (DE); Bodo KIRSCH, Thum (DE); Pablo COLORADO, Windermere, FL (US); Ashkan SHOAMANESH, Hamilton (CA); Robert HART, Hamilton (CA)

(21) Appl. No.: 18/612,570

(22) Filed: Mar. 21, 2024

Related U.S. Application Data
(60) Provisional application No. 63/454,589, filed on Mar. 24, 2023.

(51) Int. Cl. A61K 31/4439; A61K 31/4365; A61K 31/519; A61K 31/616; A61P 9/10

(52) U.S. Cl. CPC A61K 31/4439; A61K 31/4365; A61K 31/519; A61K 31/616; A61P 9/10

(57) ABSTRACT

Provided herein are methods of preventing symptomatic ischemic stroke in humans who have suffered from an acute non-cardioembolic ischemic stroke or a high-risk transient ischemic attack.

### BACKGROUND

### Field of the Disclosure

The present disclosure relates generally to methods of preventing symptomatic ischemic stroke, and more specifically to methods of preventing symptomatic ischemic stroke in humans who have suffered from an acute non-cardioembolic ischemic stroke or a high-risk transient ischemic attack.

### Background of the Disclosure

Strokes, which are among the major causes of morbidity, mortality, and long-term disability, typically refer to an acute episode of focal or global neurological dysfunction caused by an injury of the brain, spinal cord, or retina as a result of hemorrhage or infarction (BMC Neurol., 2020, 20:224). There are two major types of strokes: hemorrhagic stroke and ischemic stroke. Hemorrhagic stroke are generally caused by an acute, atraumatic extravasation of blood into the brain parenchyma, intraventricular or subarachnoid space, while ischemic stokes typically result from the occlusion of the artery that supplies blood to or within the brain. Ischemic stroke is the most frequent type of stroke, comprising on average between 80 and 90% of all strokes. During ischemic stroke, the occlusion decreases blood flow and oxygen to the brain, contributing to harm or death of brain cells. If the circulation is not reestablished quickly, the brain damage can be permanent. The severity of ischemic stroke ranges from clinically mild (i.e., a minor stroke or transient ischemic attack [TIA]) to very severe (i.e., a major ischemic stroke), but the underlying causes are nearly identical (CMAJ., 2015, 187:887-93). The initial manifestations of ischemic stroke and TIA are often followed by recurrent vascular events, including recurrent strokes (Lancet Neurol., 2017, 16:P301-10).

Non-cardioembolic ischemic strokes account for 75% of all ischemic strokes (Stroke 2021; 52; e364-467). Guideline-recommended antithrombotic prophylaxis of or standard of care for patients who have non-cardioembolic ischemic stroke includes long-term single antiplatelet therapy, sometimes after short-term dual antiplatelet therapy. However, the recurrence rate of non-cardioembolic ischemic stroke or a high-risk transient ischemic attack (TIA) remains substantial, averaging more than 6% in the year after stroke, despite the guideline-recommended treatment. Therefore, more effective methods of prevent symptomatic ischemic stroke are an important unmet need.

### BRIEF SUMMARY

In some aspects, provided is a method of preventing symptomatic ischemic stroke in a human in need thereof, wherein the human has suffered an acute non-cardioembolic ischemic stroke or a high-risk transient ischemic attack. In some embodiments, the method comprises administering to the human (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide (Compound A), which has the structure:

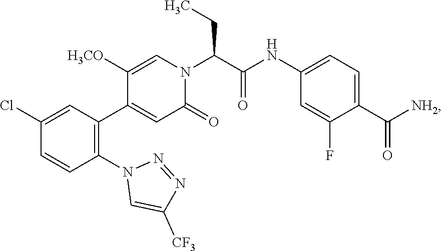 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]

or a pharmaceutically acceptable salt or solvate thereof, in a therapeutically effective amount to prevent ischemic stroke in the human. In some variations, the Compound A, or a pharmaceutically acceptable salt or solvate thereof, is administered on top of a suitable antiplatelet standard of care therapy.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

In some aspects, provided is a method of preventing symptomatic ischemic stroke in a human in need thereof, by administering to the human (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide (Compound A):

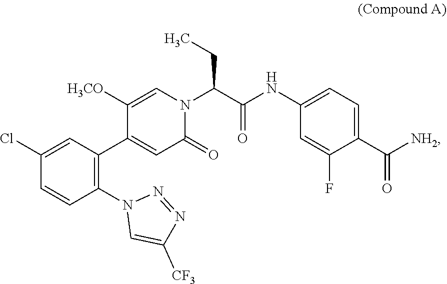 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]

or a pharmaceutically acceptable salt or solvate thereof, in a therapeutically effective amount. In some embodiments of the foregoing, the human has suffered an acute non-cardioembolic ischemic stroke or a high-risk transient ischemic attack. In some embodiments of the foregoing, the human is also administered an antiplatelet standard of care therapy.

To date, no clinical studies have established a benefit of anticoagulation for secondary prevention in human with non-cardioembolic ischemic stroke.

### Symptomatic Ischemic Stroke

In some embodiments, humans with symptomatic ischemic stroke can have rapid onset (or present on awakening) of a new focal neurological deficit with clinical (>24 hours symptoms/signs) or imaging evidence of infarction that is not attributable to a non-ischemic cause (e.g., not associated with infection, tumor, seizure, severe metabolic disease or related to a hemorrhage). In some embodiments, humans with symptomatic ischemic stroke can experience acute worsening of an existing focal neurological deficit (e.g., the qualifying stroke) that is judged to be attributable to a new infarction or extension of the previous infarction in the same vascular territory, based on brain imaging and persisting symptoms/signs of infarction and no evidence of a non-ischemic etiology. In some embodiments, humans with symptomatic ischemic stroke experience persistent, significant (worsening of NIHSS score of 4 or more), and sustained (duration of ≥24 hours or until death) symptoms/signs, if brain imaging is inconclusive or not done.

### Methods of Prevention

In some variations, the method to prevent symptomatic ischemic stroke refers to the avoidance or reduction of the risk of experiencing, suffering from or having symptomatic ischemic stroke; a condition, a disorder, a disability, an injury or a health problem associated with symptomatic ischemic stroke; the development or advancement of states of symptomatic ischemic stroke, and/or the symptoms of ischemic stroke. Such symptoms of ischemic stroke may include, for example, hemiplegia, weakness in arm, leg or face, dizziness, confusion, ataxia, dysarthria, and/or loss of vision. The treatment or prevention of a disease, a condition, a disorder, an injury or a health problem may be partial or complete.

In certain embodiments, the method provided herein decreases the likelihood of occurrence of recurrent symptomatic ischemic stroke in the human in need thereof. In some embodiments, the method provided herein decreases the likelihood of occurrence of symptomatic ischemic stroke, symptomatic stroke, cardiovascular (CV) death, all-cause mortality, myocardial infarction, systemic embolism, disabling stroke, transient ischemic attack, or a combination thereof, in the human in need thereof. In some embodiments, the method provided herein decreases the severity of ischemic stroke or stroke as assessed by modified Rankin Score (mRS) in the human in need thereof.

In some embodiments, among the human who has suffered an acute non-cardioembolic ischemic stroke or a high-risk TIA, the occurrence of symptomatic ischemic stroke in a human treated with the method provided herein is reduced by at least 10% (e.g., at least 20%, at least 30%, at least 40%, or at least 50%) compared to a human treated with placebo on top of standard of care. In some embodiments, the risk of symptomatic ischemic stroke in a human who has suffered an acute non-cardioembolic ischemic stroke or a high-risk TIA is reduced by at least 10% (e.g., at least 20%, at least 30%, at least 40%, or at least 50%) when treated with the method provided herein compared to treated with placebo on top of standard of care. In some embodiments, there are statistically significant less ischemic strokes in humans having acute non-cardioembolic ischemic stroke or a high-risk TIA with the administration of compound A, or a pharmaceutically acceptable salt thereof, or a solvate thereof, and standard of care compared to administration of placebo and standard of care with a p value <0.05.

In some embodiments, the human treated with the method provided herein has a decreased likelihood of occurrence of recurrent symptomatic ischemic stroke compared to a human treated with placebo on top of standard of care. In some embodiments, the human treated with the method provided herein has a decreased likelihood of occurrence of symptomatic ischemic stroke, symptomatic stroke, cardiovascular (CV) death, all-cause mortality, myocardial infarction, systemic embolism, disabling stroke, transient ischemic attack, or a combination thereof, compared to a human treated with placebo on top of standard of care. In some embodiments of the foregoing, the likelihood is decreased by at least 10% (e.g., at least 20%, at least 30%, at least 40%, or at least 50%).

In some embodiments, the human treated with the method provided herein has a decrease in severity of ischemic stroke or stroke as assessed by modified Rankin Score (mRS) compared to a human treated with placebo on top of standard of care.

### Pre-Existing Conditions in Human

The method provided herein is directed to treating a human in need thereof. The human in need thereof is typically a patient and does not encompass a healthy volunteer who has not suffered from an acute non-cardioembolic ischemic stroke or a high-risk transient ischemic attack.

In some embodiments, “acute non-cardioembolic ischemic stroke” typically refers to the type of ischemic stroke caused by a blood clot that formed outside the heart. Ischemic strokes or transient ischemic attacks typically result from a blocked or reduced blood flow to a part of the brain, which are further caused by blood clots that travel to the brain and block the vessels that supply it. If these blood clots form elsewhere than in the heart, the stroke is called “non-cardioembolic.” People who already had a non-cardioembolic stroke are more likely to have another stroke, and the current standard of care to prevent recurrent of ischemic stroke is antiplatelet therapy.

In some embodiments, “transient ischemic attack (TIA)”, as used herein, refers to abrupt onset of a focal neurological deficit attributed to brain or retinal ischemia with resolution of symptoms and signs within 24 h and without neuroimaging evidence of acute infarction. In some embodiments, “high risk TIA” refers to the type of TIA that has a higher risk of developing or suffering from an ischemic stroke. A measure to determine the risk is the ABCD2 score. Scores of 6 or 7 identify patients at high risk.

In some embodiments, the human has or is suffering from atherosclerosis as determined by vascular imaging. In some embodiments, the human has or is suffering from atherosclerosis based on the human's medical history. In some embodiments, the human has suffered from a large or cortical stroke as determined by brain imaging. In some embodiments, the human (i) has or is suffering from atherosclerosis as determined by vascular imaging, (ii) has or is suffering from atherosclerosis based on the human's medical history, and/or (iii) has suffered from a large or cortical stroke as determined by brain imaging.

In some variations, the human has or is suffering from atherosclerosis as determined by vascular imaging. In some variations, the human has or is suffering from atherosclerosis as determined by vascular imaging by computed tomography angiography, magnetic resonance angiography, ultrasound and/or digital subtraction angiography. In certain variations, the human has or is suffering from cerebrovascular atherosclerosis as defined by vascular imaging showing atherosclerotic plaque involving intracranial or extracranial cerebral arteries or the aortic arch. In one variation, the vascular imaging involves computed tomography angiography, magnetic resonance angiography, ultrasound and/or digital subtraction angiography. In other variations, the human has or is suffering from atherosclerosis based on the human's medical history. In one variation, the human has: a) coronary artery disease or prior acute myocardial infarction with documented coronary atherosclerotic disease, prior coronary artery bypass graft, or prior percutaneous coronary intervention; b) peripheral artery disease requiring previous bypass surgery, or percutaneous transluminal angioplasty revascularization, limb or foot amputation for arterial vascular disease, or history of intermittent claudication and one or more of the following: 1) an ankle/arm blood pressure (BP) ratio <0.90, or 2) documented peripheral artery stenosis; c) Carotid stenosis or previous carotid revascularization; or d) Documented aortic plaque, or any combination of a)-d).

In other variations, the human has suffered from a large or cortical stroke as determined by brain imaging. In certain variations, brain imaging of the human has demonstrated an acute non-lacunar infarct (computed tomography, computed tomography perfusion or diffusion weighted imaging magnetic resonance imaging) defined as cortical location and/or size >20 mm for diffusion weighted imaging and >15 mm for computed tomography.

### Compound A and Pharmaceutical Compositions Thereof

In some embodiments, the method comprises administering to the human in need thereof Compound A, or a pharmaceutically acceptable salt thereof, or a solvate thereof, on top of the antiplatelet standard of care therapy. Compound A is (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide, and has the following structure:

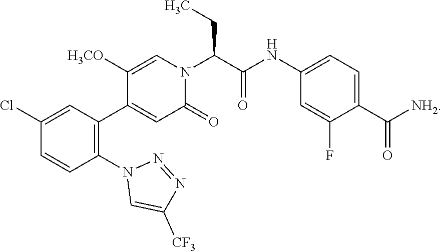 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]

Compound A can be prepared according to any suitable method known in the art, including according to the method as described in Example 235 of WO2017/005725 and Working Examples between pages 23 to 28 of WO 2019/175043, which are hereby incorporated by reference with respect to the synthesis and characterization of the compound.

In some embodiments, Compound A, as used herein, also intends to encompass all suitable isotopic variants of it. An isotopic variant of Compound A is understood here as meaning a compound in which at least one atom within the inventive compound has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, fluorine, and chlorine, such as ²H (deuterium), ³H (tritium), ¹³C, ¹⁴C, ¹⁵N, ¹⁷O, ¹⁸O, ¹⁸F, or ³⁶Cl.

In some embodiments, the method comprises administering to the human a pharmaceutical acceptable salt of Compound A on top of the antiplatelet standard of care therapy.

In some variations, physiologically acceptable salts of Compound A include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

In other variations, physiologically acceptable salts of Compound A also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monocthanolamine, diethanolamine, triethanolamine, dicyclohexylamine, dimethylaminocthanol, procaine, dibenzylamine, N-methylmorpholine, arginine, lysine, ethylenediamine, N-methylpiperidine and choline.

In some embodiments, provided herein is a method of preventing ischemic stroke in a human in need thereof, comprising administering to the human a pharmaceutical composition comprising Compound A, or a pharmaceutically acceptable salt thereof, or a solvate thereof, on top of the antiplatelet standard of care therapy. Suitable pharmaceutical compositions comprising Compound A, or a pharmaceutically acceptable salt thereof, or a solvate thereof, are described in WO 2022/189278, which is hereby incorporated by reference in its entirety, including specifically the paragraphs directed to the solid pharmaceutical dosage forms described therein on pages 8-13, as well as Examples 4-1 to 4-18 on pages 24-25.

In some embodiments, the pharmaceutical composition comprising Compound A, or a pharmaceutically acceptable salt thereof, or a solvate thereof, is a solid pharmaceutical dosage form for oral administration comprising an amorphous solid dispersion (ASD) containing Compound A, or a pharmaceutically acceptable salt thereof, or a solvate thereof, in a pharmaceutically acceptable matrix. In some embodiments, the pharmaceutical composition comprising Compound A, or a pharmaceutically acceptable salt thereof, or a solvate thereof, is a solid pharmaceutical dosage form for oral administration comprising an amorphous solid dispersion (ASD) containing Compound A, or a pharmaceutically acceptable salt thereof, or a solvate thereof, in a pharmaceutically acceptable matrix, and further pharmaceutical acceptable excipients. In some embodiments, the pharmaceutical composition of Compound A, or a pharmaceutically acceptable salt thereof, or a solvate thereof, is a solid pharmaceutical dosage form for oral administration comprising: a) an ASD containing Compound A, or a pharmaceutically acceptable salt thereof, or a solvate thereof, in a pharmaceutically acceptable matrix; b) at least one lubricant; c) at least one disintegration promoter;

d) optionally one or more fillers; and e) optionally one or more surfactants. In some embodiments, the pharmaceutical composition of Compound A, or a pharmaceutically acceptable salt thereof, or a solvate thereof, is able to release at least 85% of Compound A, or a pharmaceutically acceptable salt thereof, or a solvate thereof, into the release medium after 30 minutes.

In the context of the present invention, immediate release tablets are particularly those which have released at least 85% of Compound A into the release medium after 30 minutes, according to the release method of the European Pharmacopoeia, 10th Edition, last revision of monograph 01/2016, using apparatus 2 (paddle). The rotation speed of the stirrer is 75 rpm (revolutions per minute) in 900 ml release medium.

According to the present invention the release medium is acetate buffer pH 4.5+0.1% SDS or +0.15% SDS or +0.2% SDS or +0.3% SDS, or of 0.01 M hydrochloric acid+0.1% SDS or +0.2% SDS. SDS is the abbreviation for sodium dodecyl sulfate also called sodium lauryl sulfate.

In some embodiments, the pharmaceutical composition of Compound A, or a pharmaceutically acceptable salt thereof, or a solvate thereof, is a tablet. In some embodiments, the pharmaceutical composition of Compound A, or a pharmaceutically acceptable salt thereof, or a solvate thereof, is an immediate release tablet. In some embodiments, the pharmaceutical composition of Compound A, or a pharmaceutically acceptable salt thereof, or a solvate thereof, is a tablet optionally covered with a coating.

In one variation of the foregoing embodiments, the pharmaceutical composition comprises Compound A.

### Doses and Dosing Regimen

In some embodiments, Compound A, or a pharmaceutically acceptable salt thereof, or a solvate thereof, is orally administered in the form of solutions, suspensions, emulsions, lyophilizates or sterile powders.

Suitable administration forms for oral administration can comprise Compound A or a pharmaceutically acceptable salt thereof, or a solvate thereof, in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilisates, capsules (for example hard or soft gelatin capsules), sugar-coated tablets, granules, pellets, powders, emulsions, suspensions, aerosols or solutions. In one variation, Compound A, or a pharmaceutically acceptable salt thereof, or a solvate thereof, is orally administered to the human.

In certain variations, the term “effective amount” intends such amount of a compound of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or a pharmaceutically acceptable salt thereof, or a solvate thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds. In some variations, a “therapeutically effective amount” refers to an amount of a compound of the invention sufficient to produce a desired therapeutic outcome.

In some variations of the foregoing, Compound A is administered to the human. In some embodiments, Compound A is administered at a dose of about 10 mg to about 100 mg. In some embodiments, the Compound A is administered at a dose of about 20 mg. In some embodiments, the Compound A is administered at a dose of about 50 mg. In some embodiments, the Compound A is administered at a dose of about 100 mg. In some embodiments, the Compound A is administered to the human once daily. In some embodiments of the foregoing, the Compound A is orally administered to the human. In some embodiments, the Compound A is administered to the human as a chronic therapy on top of antiplatelet therapy.

### Antiplatelet Standard of Care Therapy

In the methods described herein, Compound A, or a pharmaceutically acceptable salt thereof, or a solvate thereof, is administered to the human on top of the antiplatelet standard of care therapy. In some embodiments, the Compound A, or a pharmaceutically acceptable salt thereof, or a solvate thereof, is administered to the human as a chronic therapy on top of antiplatelet standard of care therapy. In one variation of the foregoing, Compound A is administered to the human.

Standard of care is generally understood to refer to treatment that is accepted by medical experts as a proper treatment for a certain type of disease and that is widely used by healthcare professionals. In some variations of the method provided herein, the antiplatelet therapy is the standard of care for patients who have non-cardioembolic ischemic stroke. In some embodiments, antiplatelet standard of care therapy as described herein comprises administering to the human in need thereof a substance which inhibits the aggregation of platelets in a dose and dosing regimen that are accepted by medical experts as a proper antiplatelet therapy and widely used by healthcare professionals.

In some embodiments, the antiplatelet therapy comprises a single or dual antiplatelet therapy. In some embodiments, the antiplatelet therapy comprises acetylsalicylic acid, clopidogrel, ticagrelor, prasugrel, cilostazol or dipyridamole, or a combination thereof. In some embodiments, the antiplatelet therapy comprises acetylsalicylic acid. In some embodiments, the antiplatelet therapy comprises acetylsalicylic acid or clopidogrel, or a combination thereof. In some embodiments, the antiplatelet therapy comprises acetylsalicylic acid and ticagrelor. In some embodiments, the antiplatelet therapy comprises acetylsalicylic acid and cilostazol. In some embodiments, the antiplatelet therapy comprises acetylsalicylic acid and dipyridamole.

In some embodiments, the dose and dose regimen of antiplatelet therapy comprises a loading dose and one or more maintenance doses. In some embodiments, the loading dose of antiplatelet therapy is different from the maintenance dose. In some embodiments, the loading dose of antiplatelet therapy is an initial higher dose of a drug that may be given at the beginning of a course of treatment before dropping down to a lower maintenance dose. In some embodiments, the loading dose of antiplatelet therapy is given on the same day with the first administration of Compound A, or a pharmaceutically acceptable salt thereof, or a solvate thereof, as described herein. In some embodiments, the loading dose of antiplatelet therapy is given at an earlier date than the first administration of Compound A, or a pharmaceutically acceptable salt thereof, or a solvate thereof, as described herein. In some embodiments, the loading dose of antiplatelet therapy is given at a later date than the first administration of Compound A, or a pharmaceutically acceptable salt thereof, or a solvate thereof. In some embodiments, the method provided herein comprises administering to the human in need thereof both Compound A, or a pharmaceutically acceptable salt thereof, or a solvate thereof, and the maintenance dose of the antiplatelet therapy.

In some embodiments, the antiplatelet therapy comprises acetylsalicylic acid, and the acetylsalicylic acid is administered to the human in an amount of about 75 mg to 325 mg daily. In some embodiments, the acetylsalicylic acid is administered to the human in an amount of about 75 mg daily. In some embodiments, the acetylsalicylic acid is administered to the human in an amount of about 81 mg daily. In some embodiments, the acetylsalicylic acid is administered to the human in an amount of about 100 mg daily. In some embodiments, the acetylsalicylic acid is administered to the human in an amount of about 325 mg daily.

In some embodiments, the antiplatelet therapy comprises clopidogrel, and wherein clopidogrel is administered in an amount of about 75 mg to 300 mg daily. In some embodiments, the clopidogrel is administered to the human in an amount of about 75 mg daily. In some embodiments, the clopidogrel is administered to the human in an amount of about 300 mg daily. In some embodiments, the clopidogrel is administered to the human in an amount of about 300 mg daily only once as a loading dose, and is administered to the human in an amount of about 75 mg daily as a maintenance dose.

In some embodiments, the antiplatelet therapy comprises ticagrelor, and wherein ticagrelor is administered in an amount of about 60 mg to 180 mg daily. In some embodiments, the ticagrelor is administered to the human in an amount of about 180 mg daily. In some embodiments, the ticagrelor is administered to the human in an amount of about 90 mg twice daily. In some embodiments, the ticagrelor is administered to the human in an amount of about 180 mg daily as a loading dose, and is administered to the human in an amount of about 90 mg twice daily as a maintenance dose.

In some embodiments, the antiplatelet therapy comprises Prasugrel, and wherein Prasugrel is administered in an amount of about 5 mg to 60 mg daily. In some embodiments, the Prasugrel is administered to the human in an amount of about 60 mg daily. In some embodiments, the Prasugrel is administered to the human in an amount of about 5 to about 10 mg daily. In some embodiments, the Prasugrel is administered to the human in an amount of about 60 mg daily only once as a loading dose, and is administered to the human in an amount of about 5 to about 10 mg daily as a maintenance dose.

In some embodiments, the antiplatelet therapy is a dual antiplatelet therapy comprising acetylsalicylic acid and dipyridamole. In some embodiments, the antiplatelet therapy comprises acetylsalicylic acid and dipyridamole, wherein the acetylsalicylic acid is administered to the human in an amount of about 25 mg to 50 mg daily. In some embodiments, the acetylsalicylic acid is administered to the human in an amount of about 50 mg daily, for example, about 25 mg twice daily. In some embodiments, the antiplatelet therapy comprises acetylsalicylic acid and dipyridamole, wherein the dipyridamole is administered to the human in an amount of about 400 mg daily. In some embodiments of the foregoing, the dipyridamole is administered to the human in an amount of about 200 mg twice daily. In some embodiments of the foregoing, the dipyridamole is formulated as extended-releasing forms. In some embodiments, the antiplatelet therapy comprises acetylsalicylic acid and dipyridamole, wherein the acetylsalicylic acid is administered to the human in an amount of about 50 mg daily, and the dipyridamole is administered to the human in an amount of about 400 mg daily. In some embodiments, the acetylsalicylic acid is administered to the human in an amount of about 25 mg twice daily, and the dipyridamole is administered to the human in an amount of about 200 mg twice daily.

In some embodiments, the antiplatelet therapy comprises cilostazole, and the cilostazole is administered to the human in an amount of about 100 mg to 300 mg daily. In some embodiments, the cilostazole is administered to the human in an amount of about 200 mg daily. In some embodiments of the foregoing, the cilostazole is administered to the human in an amount of about 100 mg twice daily.

In some embodiments, the antiplatelet therapy is a dual antiplatelet therapy comprising clopidogrel and acetylsalicylic acid. In some embodiments, the antiplatelet therapy is a dual antiplatelet therapy comprising clopidogrel and acetylsalicylic acid, which is switched after about 21 days to about 90 days to a single antiplatelet therapy with acetylsalicylic acid or clopidogrel. In some embodiments, the antiplatelet therapy is a dual antiplatelet therapy including ticagrelor and acetylsalicylic acid. In some embodiments, the antiplatelet therapy is a dual antiplatelet therapy including ticagrelor and acetylsalicylic acid, which is switched after about 21 days to about 30 days to a single antiplatelet therapy with acetylsalicylic acid or clopidogrel.

It should be understood that the method provided herein is intended to encompass any of the antiplatelet therapies described herein, including the corresponding doses and dose regimens, in combination with any of the doses and administration methods of Compound A, or a pharmaceutically acceptable salt thereof, or a solvate thereof, as described herein.

In some embodiments, the method provided herein comprises administering to the human need thereof Compound A, or a pharmaceutically acceptable salt thereof, or a solvate thereof, on top of antiplatelet standard of care therapy, wherein the administration of the compound and antiplatelet therapy is concurrent. In some embodiments, the human receives the first dose of Compound A, or a pharmaceutically acceptable salt thereof, or a solvate thereof, and first dose of antiplatelet therapy on the same day. In some embodiments, the human received the first dose of antiplatelet therapy on a date earlier than the date when the human receives the first dose of Compound A, or a pharmaceutically acceptable salt thereof, or a solvate thereof. In some embodiments, the human received the first dose of antiplatelet therapy on a date later (e.g., about one day or two days later) than the date when the human receives the first dose of Compound A, or a pharmaceutically acceptable salt thereof, or a solvate thereof. In one variation of the foregoing embodiments, Compound A is administered to the human.

### Examples

The presently disclosed subject matter will be better understood by reference to the following Examples, which are provided as exemplary of the invention, and not by way of limitation.

### Example 1: PACIFIC-STROKE Clinical Trial

This example describes the PACIFIC-STROKE trial, which was a dose-finding Phase 2 study designed to assess the safety and potential efficacy of Compound A for prevention of brain infarction, both covert and symptomatic, in patients with acute non-cardioembolic ischemic stroke. This trial was also used to assess Compound A as a potential add-on to antiplatelet therapy.

The spectrum of brain infarction included both clinical recurrent ischemic strokes (requiring recognized symptoms or signs) and incident covert brain infarction (in the absence of recognized symptoms or signs). Covert brain infarcts typically are incident infarcts detected by serial MRI in the absence of symptoms and signs consistent with the location of the infarct on the MRI. Symptomatic ischemic stroke contrasts with covert brain infarcts at least by having recognized symptoms or signs.

### Eligibility

In this international, randomized, placebo-controlled, double-blind, parallel-group, dose-finding trial, participants were recruited from 196 hospitals in 23 countries. Individuals aged 45 years or older with non-cardioembolic ischemic stroke (with persistent signs or symptoms that lasted for at least 24 h and/or acute brain infarction documented via brain imaging) were eligible for participation within 48 h of symptom onset (or last known to be without symptoms) if their treating physician intended to treat them with antiplatelet therapy during the study period, if they had brain imaging (CT or MRI) that excluded haemorrhagic stroke or other pathology that could explain symptoms, were able to undergo a baseline MRI (either before or within 72 h after randomization), and willing to adhere to study procedures.

The key exclusion criteria were previous ischemic stroke within 30 days of index event, history of atrial fibrillation or suspicion of cardioembolic stroke, active bleeding or history of major bleeding, uncontrolled hypertension, estimated glomerular filtration rate of less than 30 mL/min per 1.73 m2, clinically significant liver disease, major surgery within 30 days before randomization or planned surgery or intervention during the study period, treatment with a strong inducer or inhibitor of cytochrome P450 isoenzyme 3A4 within 14 days of randomization, and indication for full dose and long-term anticoagulation therapy during study.

### Procedures

Participants were given either Compound A orally once daily or matched placebo for a period of 26-52 weeks. All participants were required to undergo at least two MRIs that met study requirements. In addition to the baseline MRI (obtained after the index stroke either before randomization or within 72 h after randomization), participants underwent a final study MRI at 26 weeks or as soon as possible after early termination of assigned study treatment. Patients were followed up for at least 26 weeks and up to 52 weeks and assessed for the occurrence of safety, efficacy, and adverse events at 2, 4, 8, 13, 20, 26, 32, 39, 46, and 52 (end of treatment) weeks after randomization, and again 2 weeks after the end of treatment visit. Adherence to assigned therapy was assessed by means of interview and pill count at each clinic visit.

### Outcomes

The effect of different doses of Compound A on prevention of all types of brain infarction was assessed. The spectrum of brain infarction included both clinical recurrent ischemic strokes (requiring recognized symptoms or signs) and incident covert brain infarction (detected by MRI and in the absence of recognized symptoms or signs).

The primary efficacy endpoint was the composite of symptomatic recurrent ischemic stroke and incident covert brain infarcts detected on follow-up MRI at or before 26 weeks after randomization. Incident covert brain infarcts were counted in participants without recurrent ischemic stroke before repeat MRI to avoid counting new lesions related to symptomatic infarcts.

Secondary efficacy endpoints were the composite of ischemic stroke, cardiovascular death, and myocardial infarction; symptomatic recurrent ischemic stroke; any recurrent stroke (symptomatic ischemic and haemorrhagic stroke); disabling stroke (defined as a modified Rankin Scale [mRS] score of >4); and all-cause mortality. Additionally, the secondary endpoint of the composite of recurrent symptomatic ischemic stroke, covert brain infarcts detected on MRI, cardiovascular death, myocardial infarction, and systemic embolism was reported at 26 weeks.

Systemic embolism being evaluated in this example included abrupt vascular insufficiency associated with clinical or radiological evidence of arterial occlusion in the absence of other likely mechanisms. It did not include thromboembolism of the pulmonary vasculature or venous thrombosis, e.g. pulmonary embolism or deep venous thrombosis.

Human diagnosed with myocardial infarction (MI) generally exhibited evidence of myocardial necrosis in a clinical setting consistent with acute myocardial ischemia. According to the MI Universal Definition from 2018, the diagnosis of MI requires the combination of: 1) presence of acute myocardial injury (changes in cardiac biomarkers) and 2) evidence of acute myocardial ischemia derived from the clinical presentation, electrocardiographic changes, or the results of myocardial or coronary artery imaging, or in case of post-mortem pathological findings irrespective of biomarker values.

Cardiovascular death being recorded in the trial included death due to stroke, myocardial infarction, heart failure or cardiogenic shock, sudden death or any other death due to other cardiovascular causes. In addition, death due to non-traumatic hemorrhage was also included.

The primary safety outcome was the composite of major bleeding and clinically relevant non-major bleeding, according to the criteria of the International Society on Thrombosis and Haemostasis (ISTH). Secondary safety outcomes were all bleeding, ISTH-defined major bleeding, ISTH-defined clinically relevant non-major bleeding, ISTH-defined minor bleeding, and symptomatic intracerebral haemorrhage (non-traumatic). Additional prespecified exploratory safety outcomes included haemorrhagic transformation within the qualifying stroke detected by baseline MRI performed post-study drug or on follow-up study MRIs based on the Heidelberg classification of severity (haemorrhagic infarct 1 or 2 or parenchymal haematoma 1 or 2).

Following ISTH-defined criteria, major bleeding was recorded in this trial if an event that met at least one of the below criteria happened: 1) fatal bleeding, and/or 2) symptomatic bleeding in a critical area or organ (intracranial, intraocular, intraspinal, pericardial, retroperitoneal, intraarticular, or intramuscular with compartment syndrome), and/or 3) clinically overt bleeding associated with a recent decrease in the hemoglobin level of ≥2 g/dL (20 g/L; 1.24 mmol/L) compared to the most recent hemoglobin value available before the event, and/or 4) clinically overt bleeding leading to transfusion of 2 or more units of packed red blood cells or whole blood. The “overt” bleeding required the identification of the bleeding location and the hemoglobin drop and/or transfusion needs to be related to the bleeding.

Following ISTH-defined criteria, clinically relevant non-major bleeding was recorded if any sign or symptom of hemorrhage that did not fit the criteria for the ISTH definition of major bleeding was observed, but did meet at least one of the following criteria: 1) requiring medical intervention by a healthcare professional; 2) leading to hospitalization or increased level of care; an/or 3) prompting a face to face (i.e. not just a telephone or electronic communication) evaluation.

Following ISTH-defined criteria, a minor bleeding was recorded when all other overt bleeding episodes not meeting the above criteria for ISTH major or clinically relevant non-major bleeding (e.g., bleeding from a minor wound that does not prompt a face-to-face evaluation for a physical examination or laboratory testing) happened.

Exploratory post-hoc efficacy outcomes were assessment of the effect of each Compound A dose on the occurrence of transient ischemic attack and the composite of recurrent symptomatic ischemic stroke and transient ischemic attack.

### Statistical Analysis

The primary efficacy analysis was the assessment of the overall dose-response effect of Compound A on the primary efficacy outcome at 26 weeks. All efficacy outcomes were analyzed using the intention-to-treat principle, such that the efficacy analyses included all patients randomly assigned to treatment who were alive at the time of analysis and regardless of treatment discontinuation. Exploratory analyses of treatment effects on the primary binary efficacy outcome at 26 weeks were conducted using unadjusted logistic regression models in prespecified subgroups. Post-hoc exploratory subgroup analyses confined to the effect of Compound A 50 mg daily versus placebo on the composite outcome of recurrent symptomatic ischemic stroke and transient ischemic attack (data driven) were conducted in prespecified subgroups indicating atherosclerotic disease as well based on MRI patterns (location and size).

### Results

### Participants

Between Jun. 15, 2020, and Jul. 22, 2021, of 1880 people screened, 1808 participants were randomly assigned to either Compound A 10 mg (n=455), 20 mg (n=450), or 50 mg (n=447), or placebo (n=456). End of treatment visits were performed in all ongoing patients 26 weeks after randomization of the last patient. The trial ended on Feb. 18, 2022 (last patient visit) and data base closure was on Mar. 10, 2022.

### Primary Efficacy Outcome

At 26 weeks after randomization, the primary composite efficacy outcome was observed in 87 (19%) of 456 participants in the placebo group versus 86 (19%) of 455 in the Compound A 10 mg group, 99 (22%) of 450 in the Compound A 20 mg group, and 90 (20%) of 447 in the Compound A 50 mg group. The results are summarized in Table 1-1.

With respect to the primary composite efficacy outcome, no significant difference was seen between the placebo group and any of the Compound A dose groups and no significant dose response was observed (Emax2 model t statistic-0.68; p=0.80). No significant heterogeneity was found of treatment effects in the prespecified exploratory subgroup analyses after adjustment for multiple comparisons. 275 (76%) of 362 events that comprised the composite primary outcome were incident covert brain infarcts. Incident covert brain infarcts were identified in 275 participants (MRI detected in 219 [80%] participants, imputed in 56 [20%] participants); 153 (70%) of 219 MRI-detected covert brain infarcts were small (≤15 mm in diameter) and subcortical, while 68 (31%) were cortical or large (>15 mm). The frequency of incident covert brain infarcts was similar across treatment groups.

TABLE 1-1
Efficacy outcomes

| | | | | | | |
|---|---|---|---|---|---|---|
| | Cmpd A | Cmpd A | Cmpd A | Cmpd A | Cmpd A | Cmpd A |
| | 10 mg | 10 mg | 20 mg | 20 mg | 50 mg | 50 mg |
| Placebo | group | vs | group | vs | group | vs |
| (n = 456) | (n = 455) | placebo | (n = 450) | placebo | (n = 447) | placebo |

Primary outcome

| | | | | | | | |
|---|---|---|---|---|---|---|---|
| Ischemic stroke or | 87 | 86 | 0.99 | 99 | 1.15 | 90 | 1.06 |
| covert infarcts* | (19%) | (19%) | (0.79- | (22%) | (0.93- | (20%) | (0.85- |
| | | | 1.24) | | 1.43) | | 1.32) |

Secondary outcomes
Components of the primary outcome*

| | | | | | | | |
|---|---|---|---|---|---|---|---|
| Incident covert brain | 64 | 63 | 0.99 | 74 | 1.17 | 74 | 1.17 |
| infarcts on MRI† | (14%) | (14%) | (0.75- | (16%) | (0.90- | (17%) | (0.91- |
| | | | 1.30) | | 1.51) | | 1.52) |
| Recurrent | 23 | 24 | 1.05 | 25 | 1.10 | 17 | 0.75 |
| symptomatic ischemic | (5%) | (5%) | (0.66- | (6%) | (0.69- | (4%) | (0.45- |
| stroke* | | | 1.67) | | 1.75) | | 1.26) |

Efficacy outcomes‡

| | | | | | | | |
|---|---|---|---|---|---|---|---|
| Recurrent | 28 | 26 | 0.93 | 26 | 0.94 | 22 | 0.80 |
| symptomatic ischemic | (6%) | (6%) | (0.59- | (6%) | (0.60- | (5%) | (0.50- |
| stroke§ | | | 1.45) | | 1.47) | | 1.27) |
| Any recurrent stroke§ | 30 | 26 | 0.86 | 26 | 0.88 | 25 | 0.85 |
| | (7%) | (6%) | (0.56- | (6%) | (0.56- | (6%) | (0.54- |
| | | | 1.34) | | 1.36) | | 1.32) |
| Disabling stroke (mRS | 3 | 5 | 1.67 | 5 | 1.69 | 1 | 0.34 |
| score of ≥4)§ | (1%) | (1%) | (0.50- | (1%) | (0.51- | (<1%) | (0.05- |
| | | | 5.55) | | 5.62) | | 2.27) |
| Recurrent | 35 | 33 | 0.94 | 30 | 0.87 | 33 | 0.96 |
| symptomatic ischemic | (8%) | (7%) | (0.63- | (7%) | (0.58- | (7%) | (0.64- |
| stroke, vascular death, | | | 1.40) | | 1.30) | | 1.43) |
| or myocardial | | | | | | | |
| infarction§ | | | | | | | |
| Recurrent | 79 | 80 | 0,95 | 87 | 1.06 | 81 | 1.03 |
| symptomatic ischemic | (17%) | (18%) | (0.76- | (19%) | (0.85- | (18%) | (0.82- |
| stroke, incident covert | | | 1.20) | | 1.33) | | 1.30) |
| brain infarct on MRI, | | | | | | | |
| cardiovascular death, | | | | | | | |
| myocardial infarction | | | | | | | |
| and systemic | | | | | | | |
| embolism* | | | | | | | |
| All-cause mortality§ | 10 | 10 | 1.00 | 6 | 0.60 | 17 | 1.72 |
| | (2%) | (2%) | (0.48- | (1%) | (0.26- | (4%) | (0.89- |
| | | | 2.09) | | 1.41) | | 3.32) |

Post-hoc exploratory outcomes‡

| | | | | | | | |
|---|---|---|---|---|---|---|---|
| Transient ischemic | 11 | 10 | 0.91 | 2 | 0.18 | 2 | 0.18 |
| attack | (2%) | (2%) | (0.44- | (<1%) | (0.05- | (<1%) | (0.05- |
| | | | 1.87) | | 0.64) | | 0.65) |
| Recurrent | 38 | 35 | 0.92 | 28 | 0.74 | 24 | 0.64 |
| symptomatic ischemic | (8%) | (8%) | (0.63- | (6%) | (0.49- | (5%) | (0.41- |
| stroke or transient | | | 1.35) | | 1.12) | | 0.98) |
| ischemic attack | | | | | | | |

Data are n (%) or hazard ratio with 90% CI in parentheses, or crude incidence ratio with 90% CI in parentheses. The cause-specific Cox proportional hazard model is modelled on the basis of the time to first occurrence of the event. The independent variable is treatment. Hazard ratios are calculated separately for all comparisons.
mRS = modified Rankin Scale.
*Proportion of outcomes at 26 weeks and accompanying crude incidence ratios for these binary event outcomes are presented.
†Incident covert brain infarct data missing in 352 patients and imputed in patients who did not otherwise meet the primary efficacy outcome based on having a symptomatic recurrent ischemic stroke.
‡Hazard ratios calculated using Cox proportional hazard model are presented.
§Proportion of outcomes at end study.

### Other Efficacy Outcome and Post-Hoc Analysis

Observations with respect to other efficacy outcomes and post-hoc analysis results are also shown in Table 1-1. As of data cutoff, 102 recurrent symptomatic ischemic strokes occurred: 28 (6%) in the placebo group, 26 (6%) in the Compound A 10 mg group, 26 (6%) in the 20 mg group, and 22 (5%) in the 50 mg group. Prespecified secondary efficacy outcomes are presented in Table 1-1, with no significant differences seen between the placebo group and any of the Compound A dose groups.

However, surprisingly, in post-hoc analysis, the frequency of the composite outcome of recurrent ischemic stroke and transient ischemic attack was much lower among participants assigned to Compound A than among participants assigned to placebo, particularly among those assigned to Compound A 50 mg, as shown in Table 1-1.

The primary safety composite outcome of ISTH major and clinically relevant non-major bleeding occurred while on treatment in 11 (2%) of 452 participants taking placebo, 19 (4%) of 445 taking Compound A 10 mg, 14 (3%) of 446 taking Compound A 20 mg, and 19 (4%) of 443 taking Compound A 50 mg. No dose-response association and no significant increase was found in the proportion of primary safety outcome events in the pooled Compound A groups compared with the proportion of events in the placebo group (HR 1.57 [90% CI 0.91-2.71]). Primary intracerebral haemorrhages occurred in three (1%) participants who received Compound A 50 mg daily and one (<1%) who received placebo (HR 3.05 [90% CI 0.46-20.4]). On baseline MRIs done after study drug initiation, there was no increase in secondary haemorrhagic transformation of the index stroke resulting in haemorrhagic infarcts, including incident parenchymal haematoma 1 or parenchymal haematoma 2 among those assigned to Compound A.

As discussed above, in this international, dose-finding trial of patients with acute non-cardioembolic ischemic stroke receiving antiplatelet therapy, we found no overall dose-response effect with Compound A on the primary efficacy composite outcome of recurrent ischemic stroke and incident covert brain infarcts. This finding was in part accounted for by the absence of reduction of incident covert brain infarcts that contributed 75% of primary outcome events.

However, surprisingly, in post-hoc analyses, treatment with Compound A 50 mg daily reduced the clinical outcome of recurrent symptomatic ischemic stroke and transient ischemic attack, and this reduction was pronounced among participants with coexistent atherosclerosis. Although the primary and secondary efficacy endpoints were negative, post-hoc analyses suggested that Compound A was effective for prevention of the composite of recurrent ischemic stroke and transient ischemic attack when added to antiplatelet therapy in patients with acute non-cardioembolic ischemic stroke associated with atherosclerosis without causing new safety signals.

### Subgroup Analysis

Analysis of the trial results also indicated a consistent trend in benefit for patients with evidence of cerebrovascular or systemic atherosclerosis. As shown in Table 1-2, symptomatic ischemic stroke was reduced for the 50 mg dose of Compound A in patient subgroups with atherosclerosis in the medical history (HR: 0.703 [90% CI: 0.348-1.421]), based on the investigator reported TOAST classification of large artery disease stroke (HR: 0.519 [90% CI:0.234-1.151]), large artery atherosclerosis on vascular imaging with CTA, MRA or carotid ultrasound (HR 0.515 [90% CI: 0.226-1.173]), and large or cortical lesions on brain MRI (HR 0.649 [90% CI: 0.374-1.127]). On the contrary, patients with only a single small subcortical lacunar lesion in the MRI did not show any benefit (HR: 1.442 [90% CI: 0.550-3.780]). This potentially also explained why no effect on the primary efficacy endpoint (composite of symptomatic ischemic stroke and covert brain infarcts) was seen, as covert brain infarcts were primarily focal subcortical lesions associated with small vessel disease (Longstreth et al. 2002, Osman et al. 2018), for which no benefit could be shown.

TABLE 1-2
Subgroup analyses: number of participants with recurrent
symptomatic ischemic stroke, PACIFIC trial (FAS)

| | | | | | Compound A |
|---|---|---|---|---|---|
| | | | | | 50 mg vs |
| | Compound A | | | | Placebo |
| | 50 mg | | Placebo | | Cause- |
| | | IR | | IR | specific HR |
| | n/N | (90% | n/N | (90% | (90% |
| Subgroups | (%) | CI) | (%) | CI) | CI) |
| Large artery | 7/89 | 11.13 | 11/76 | 21.19 | 0.519 |
| atherosclerosis | (7.86%) | (5.22- | (14.47%) | (11.8- | (0.234- |
| (TOAST) | | 18.83) | | 32.67) | 1.151) |
| Atherosclerosis | 9/114 | 10.78 | 14/119 | 15.45 | 0.703 |
| by medical | (7.89%) | ((5.62- | (11.76%) | (9.34- | (0.348- |
| history^{b} | | 17.29) | | 22.81) | 1.421) |
| Atherosclerosis | 6/195 | 4.15 | 12/198 | 7.87 | 0.515 |
| by vascular | (3.07%) | 1.81- | (6.06%) | (4.54- | (0.226- |
| imaging | | 7.26) | | 11.94) | 1.173) |
| Large or | 15/272 | 7.33 | 22/265 | 11.33 | 0.649 |
| Cortical stroke | (5.51%) | (4.52- | (8.30%) | (7.67- | (0.374- |
| (MRI) | | 10.70) | | 15.57) | 1.127) |
| No large or | 7/141 | 6.54 | 5/146 | 4.17 | 1.442 |
| cortical | (4.96%) | (3.07- | (3.42%) | (1.64- | (0.550- |
| stroke (MRI) | | 11.07) | | 7.63) | 3.780) |

FAS = full analysis set,
HR = hazard ration;
IR = incidence rate,
^{a} detected and imputed
^{b}Atherosclerosis based on the medical history (CAD/MI, PAD, Carotid artery disease, Vascular procedures [PTCA, PCI, CABG]).
For composite outcomes and each component, the first event after randomization was considered. Subsequent events of the same type were not shown

### Example 2: OCEANIC-STROKE Clinical Trial

Further to this subgroup analysis in Example 1, a new trial was designed, which includes the patient groups with “Atherosclerosis by medical history”, “Atherosclerosis by vascular imaging”, and “Large or Cortical stroke (MRI)” shown in Table 1-2, and therefore, restricts the patient population, especially excludes patients with no large or cortical stroke and thus, isolated small subcortical strokes (last row of Table 1-2).

With this sub-patient group selection, this new trial is better to explore whether Compound A works better than placebo at reducing ischemic strokes in participants who recently had a non-cardioembolic ischemic stroke or high-risk TIA when given in addition to standard antiplatelet therapy.

A multicenter, international, randomized, placebo controlled, double-blind, parallel group and event driven trial is conducted. The trial seeks to study the efficacy of Compound A for the prevention of ischemic stroke in male and female participants aged 18 years and older after an acute non-cardioembolic ischemic stroke or high-risk TIA. Another aim of this trial is to compare the occurrence of major bleeding events during the study between the Compound A and the placebo group. Major bleedings have a serious or even life-threatening impact on a person's health.

Dependent on the treatment group, the participants either take 50 mg Compound A or placebo as oral tablets once a day for around 3 months up to 31 months. Both Compound A and placebo are administered orally once daily. The participants also receive standard of care, antiplatelet therapy concurrently. The antiplatelet therapy include single or dual antiplatelet therapy including ASA, clopidogrel, ticagrelor, prasugrel, cilostazol and dipyridamole and in line with local guidelines. During the study, vital signs such as blood pressure and heart rate of the participants are regularly checked. The participants' heart health is examined using an electrocardiogram (ECG). Blood samples of the participants are collected. Approximately every 3 months during the treatment period, either a phone call or a visit to the study site is scheduled on an alternating basis. In addition, one visit before and up to two visits after the treatment period is scheduled if necessary.

### Eligibility

The ages eligible for this study is 18 years old and order, and all sexes are eligible for this study. The patients must have experienced acute non-cardioembolic ischemic stroke or high-risk TIA and in addition must have systemic or cerebrovascular atherosclerosis or acute non-lacunar infarct.

The patient group may be further restricted to patients who have at least one of the following criteria a to c:
a. Cerebrovascular atherosclerosis defined as vascular imaging (CTA, MRA, ultrasound, DSA) showing atherosclerotic plaque involving intracranial or extracranial cerebral arteries or the aortic arch,
OR
b. Medical history of atherosclerosis:
   i. CAD or AMI with documented coronary atherosclerotic disease, prior CABG, or prior PCI
   ii. PAD requiring previous bypass surgery, or percutaneous transluminal angioplasty revascularization, limb or foot amputation for arterial vascular disease (i.e., excludes trauma), OR history of intermittent claudication and one or more of the following: 1) an ankle/arm blood pressure (BP) ratio <0.90, or 2) documented peripheral artery stenosis
   iii. Carotid stenosis ≥50% or previous carotid revascularization
   iv. Documented aortic plaque,
OR
c. Brain imaging demonstrating an acute non-lacunar infarct (CT, CT perfusion or DWI MRI) defined as cortical location and/or size >20 mm for DWI and >15 mm for CT.

Patients with ischemic stroke≤7 days before the index stroke event are excluded from the study. Also excluded are patients with index stroke following procedures or strokes due to other rare causes, and/or with history of atrial fibrillation/flutter, left ventricular thrombus, mechanic valve or other cardioembolic source of stroke requiring anticoagulation.

### Outcomes

The primary efficacy outcome for the randomized comparison between Compound A and placebo is the time to first occurrence of symptomatic ischemic stroke. The main safety outcome measure is time to first occurrence of ISTH major bleeding (ISTH=International Society on Thrombosis and Hemostasis). The time frame is up to about 31 months.

15 secondary outcome measures are used, including: 1) time to first occurrence of all strokes (ischemic and hemorrhagic); 2) time to first occurrence of composite of cardiovascular (CV) death, myocardial infarction (MI) or stroke; 3) time to first occurrence of composite of all-cause mortality, MI or stroke; 4) time to first occurrence of disabling stroke (Modified Rankin Scale (mRS) ≥3 at 90 days); 5) time to first occurrence of all-cause mortality; 6) time to first occurrence of transient ischemic attack (TIA); 7) time to first occurrence of composite of ISTH major or clinically relevant non-major bleeding; 8) time to first occurrence of ISTH clinically relevant non-major bleeding; 9) time to first occurrence of symptomatic intracranial hemorrhage; 10) time to first occurrence of hemorrhagic stroke; 11) time to first occurrence of fatal bleeding; 12) time to first occurrence of minor bleeding; 13) time to first occurrence of composite of ischemic stroke or ISTH major bleeding; 14) 5ime to first occurrence of composite of CV death, all stroke, MI or ISTH major bleeding; and 15) time to first occurrence of composite of all-cause mortality, disabling stroke, fatal bleeding, symptomatic intracranial hemorrhage. Time frames are all up to 31 months.

We claim:

1. A method of preventing symptomatic ischemic stroke in a human in need thereof, wherein the human has suffered an acute non-cardioembolic ischemic stroke or a high-risk transient ischemic attack (TIA), the method comprising administering to the human (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide (Compound A): 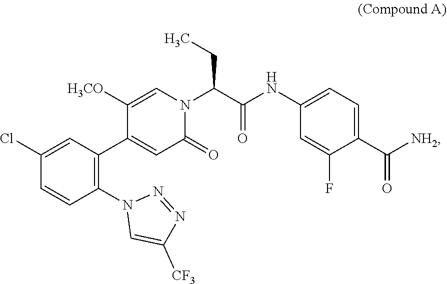 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]
or a pharmaceutically acceptable salt or solvate thereof, in a therapeutically effective amount on top of antiplatelet standard of care therapy to prevent ischemic stroke in the human.

2. The method of claim 1, wherein (i) the human has or is suffering from atherosclerosis as determined by vascular imaging, (ii) the human has or is suffering from atherosclerosis based on the human's medical history, and/or (iii) the human has suffered from a large or cortical stroke as determined by brain imaging.

3. The method of claim 1, wherein the human has or is suffering from atherosclerosis as determined by vascular imaging.

4. The method of claim 3, wherein the human has or is suffering from atherosclerosis as determined by vascular imaging by computed tomography angiography, magnetic resonance angiography, ultrasound and/or digital subtraction angiography.

5. The method of claim 3, wherein the human has or is suffering from cerebrovascular atherosclerosis as defined by vascular imaging showing atherosclerotic plaque involving intracranial or extracranial cerebral arteries or the aortic arch.

6. The method of claim 5, wherein the vascular imaging involves computed tomography angiography, magnetic resonance angiography, ultrasound and/or digital subtraction angiography.

7. The method of claim 1, wherein the human has or is suffering from atherosclerosis based on the human's medical history.

8. The method of claim 7, wherein the human has suffered:
a) coronary artery disease or acute myocardial infarction with documented coronary atherosclerotic disease, prior coronary artery bypass graft, or prior percutaneous coronary intervention;
b) peripheral artery disease requiring previous bypass surgery, or percutaneous transluminal angioplasty revascularization, limb or foot amputation for arterial vascular disease, or history of intermittent claudication and one or more of the following: 1) an ankle/arm blood pressure (BP) ratio <0.90, or 2) documented peripheral artery stenosis;
c) Carotid stenosis or previous carotid revascularization; or
d) Documented aortic plaque or any combination of a)-d).

9. The method of claim 1, wherein the human has suffered from a large or cortical stroke as determined by brain imaging and thus, has not suffered from an isolated small subcortical stroke.

10. The method of claim 9, wherein brain imaging of the human has demonstrated an acute non-lacunar infarct (computed tomography, computed tomography perfusion or diffusion weighted imaging magnetic resonance imaging) defined as cortical location and/or size >20 mm for diffusion weighted imaging and >15 mm for computed tomography.

11. The method of any one of claim 1, wherein the antiplatelet therapy comprises a single or dual antiplatelet therapy.

12. The method of any one of claim 1, wherein the antiplatelet therapy comprises acetylsalicylic acid, clopidogrel, ticagrelor, prasugrel, cilostazol or dipyridamole, or a combination thereof.

13. The method of claim 12, wherein the antiplatelet therapy comprises acetylsalicylic acid.

14. The method of claim 12, wherein the antiplatelet therapy comprises acetylsalicylic acid or clopidogrel, or a combination thereof.

15. The method of claim 12, wherein the antiplatelet therapy comprises acetylsalicylic acid and ticagrelor.

16. The method of any one of claim 1, wherein the Compound A is administered at a dose of about 10 mg to about 100 mg.

17. The method of any one of claim 1, wherein the Compound A is administered at a dose of about 20 mg.

18. The method of any one of claim 1, wherein the Compound A is administered at a dose of about 50 mg.

19. The method of any one of claim 1, wherein the Compound A is administered at a dose of about 100 mg.

20. The method of any one of claim 1, wherein the Compound A is administered to the human once daily.

21. The method of any one of claim 1, wherein the Compound A is orally administered to the human.

22. The method of claim 13, wherein acetylsalicylic acid, is administered in an amount of about 75 mg to 325 mg daily.

23. The method of claim 13, wherein acetylsalicylic acid, is administered in an amount of about 75 mg daily.

24. The method of claim 13, wherein acetylsalicylic acid, is administered in an amount of about 81 mg daily.

25. The method of claim 13, wherein acetylsalicylic acid, is administered in an amount of about 100 mg daily.

26. The method of claim 13, wherein acetylsalicylic acid, is administered in an amount of about 325 mg daily.

27. The method of claim 1, wherein the antiplatelet therapy comprises clopidogrel, and the clopidogrel is administered in an amount of about 75 mg to 300 mg daily.

28. The method of claim 27, wherein the clopidogrel is administered in an amount of about 75 mg daily.

29. The method of claim 27, wherein the clopidogrel is administered in an amount of about 300 mg daily.

30. The method of claim 27, wherein the clopidogrel is administered in an amount of about 300 mg daily as a loading dose and 75 mg daily as a maintenance dose.

31. The method of any one of claim 1, wherein the Compound A is administered to the human as a chronic therapy on top of antiplatelet therapy.

32. The method of any one of claim 1, wherein the antiplatelet therapy is a dual antiplatelet therapy comprising clopidogrel and acetylsalicylic acid, and wherein the dual antiplatelet therapy is switched after about 21 days to about 90 days to a single antiplatelet therapy comprising acetylsalicylic acid or clopidogrel.

33. The method of any one of claim 1, wherein the antiplatelet therapy is a dual antiplatelet therapy comprising ticagrelor and acetylsalicylic acid, and wherein the dual antiplatelet therapy is switched after about 21 days to about 30 days to a single antiplatelet therapy comprising acetylsalicylic acid or clopidogrel.

34. The method of any one of claim 1, wherein the method decreases the likelihood of occurrence of recurrent symptomatic ischemic stroke in the human.

35. The method of any one of claim 1, wherein the method decreases the likelihood of occurrence of symptomatic ischemic stroke, symptomatic stroke cardiovascular (CV) death, all-cause mortality, myocardial infarction, systemic embolism, disabling stroke, transient ischemic attack, or a combination thereof, in the human.

36. The method of any one of claim 1, wherein the method decreases the severity of ischemic stroke or stroke as assessed by modified Rankin Score (mRS) in the human.
